(19) United States
(12) Patent Application Publication
(10) Pub. No.: US 2011/0270234 A1
(43) Pub. Date: Nov. 3, 2011

(54) ENDOSCOPIC SURGICAL INSTRUMENT

(75) Inventors: Michael White, Leeds (GB); Vhairi Maxwell, Leeds (GB)

(73) Assignee: SURGICAL INNOVATIONS LIMITED, Leeds (GB)

(21) Appl. No.: 13/054,526

(22) PCT Filed: Jul. 31, 2009
(86) PCT No.: PCT/GB2009/050952

§ 371 Date: Apr. 29, 2011

(30) Foreign Application Priority Data
Jul. 31, 2008 (GB) 0813990.9
Oct. 3, 2008 (GB) 0818111.7
Mar. 18, 2009 (GB) 0904602.0
Apr. 9, 2009 (GB) 0906184.7

(51) Int. Cl. A61B 17/00

(52) U.S. Cl. 606/1

(57) ABSTRACT

A series of integrally formed segments 24 are allowed to move relative to each other by flexure of hinges 26 that are also integrally formed with the sections.

The present invention relates to endoscopic surgical instruments, endoscopic surgical items and surgical items, gastric bands and methods relating thereto. The instruments are particularly, but not exclusively relevant to instruments that are able to snare gastric bands or sutures.

Endoscopic surgical instruments are widely, though not exclusively, used in abdominal surgery. In abdominal surgery it is possible for a surgeon to examine or operate on abdominal organs without the physical insertion of the surgeons fingers. This is done by making a hole through the skin and other tissues in order to enter a body cavity, blowing inert gas under pressure into the abdominal cavity through that hole to create space, and inserting an endoscopic camera through that opening so that the surgeon can view the organs on a remote monitor. Other tubes are then placed in the abdominal wall (usually of 5-11 mm diameter), which are sealed to prevent or restrict the egress of the inert gas, and surgical instruments can then be inserted through these tubes. The surgical instruments can either be for operating on the organs or for pushing organs out of the way, and the surgeon is able to manipulate them from outside the body.

EP 0 623 004 (McMahon and Moran) discloses an endoscopic surgical instrument comprising an elongate portion made from a plurality of separate segments. The segments are threaded onto two parallel wires which are located towards opposed sides of the segments. The surgeon operates the endoscopic surgical instrument by operating a handle attached to the elongate portion. By tensioning the wires the surgeon is able to cause the segments to bend towards each other. The two wires are pulled simultaneously to prevent misalignment of the segment faces, As the wires are further tensioned, faces on adjacent segments abut to create a rigid hook.

There are many other publications that also disclose endoscopic surgical instruments that include a plurality of segments that can be manipulated. However, in all these documents the segments are either separate from each other, or are pivotally connected to each other. These include U.S. Pat. No. 2,079,233; U.S. Pat. No. 3,109,286; U.S. Pat. No. 3,642,352; U.S. Pat. No. 3,799,151; U.S. Pat. No. 3,958,576; U.S. Pat. No. 4,226,228; U.S. Pat. No. 4,239,036; U.S. Pat. No. 4,483,562; U.S. Pat. No. 4,754,909; U.S. Pat. No. 4,950,273; U.S. Pat. No. 4,982,727; U.S. Pat. No. 5,035,248; U.S. Pat. No. 5,152,779 DE 4 021 153; EP1 815 801; DE4 243 715; DE1960 8809; DE1992 8272; EP1 836 949; FR2 713 492; U.S. Pat. No. 4, 353,358; U.S. Pat. No. 5, 501,654; U.S. Pat. No. 5,669,926 and EP1 864 625, US 2008/051802; WO2006/057702; US 2005/273085; U.S. Pat. No. 5,448,989; WO 2006/083306; US 2005/065397 and U.S. Pat. No. 5,772,578

There are also patent publications that relate to the gripping of sutures or needles by the instruments during endoscopic surgery. These include U.S. Pat. No. 5,951,587; U.S. Pat. No. 1,445,348; U.S. Pat. No. 3,408,554; U.S. Pat. No. 5,304,185; U.S. Pat. No. 5,413,583; U.S. Pat. No. 5,601,575; U.S. Pat. No. 5,817,111 and U.S. Pat. No. 5,449,366. However, where these publications attempt to catch a suture, they are difficult to operate and manipulate. Further, the suture may snag on other parts of the instrument or on the body.

EP 1 829 505 discloses a gastric band having a suture for engagement with an instrument.

It is an object of the present invention to attempt to overcome at least one of the above or other problems.

According to one aspect of the present invention an endoscopic surgical instrument and a surgical device are provided, in which one of the instrument or device includes an opening at and end region and a channel extending from the opening towards the end of the other of the instrument or device includes an end portion arranged, in use, to be at least partially located and held in the opening and a part arranged, in use, to be at least partially located in the channel.

The part may be arranged to be completely within the channel.

The end portion may be arranged to be completely within the opening.

At least one of the opening and end portion or the channel and part may be arranged to be a loose fit.

At least one of the opening and end portion or the channel and part may be arranged to be a tight fit.

At least one of the opening and end portion or the channel and the part may be arranged to be friction fit.

Relative rotation of the instrument and the item may be arranged to occur and the relative rotation may be arranged to be about an elongate axis of the instrument. The end portion may be part spherical. The opening may be part spherical. The opening may extend across the instrument or item.

The end portion may be a bar.

The bar may be a T-shaped bar.

The end portion may be arranged to cooperate with the opening by relative sideways movement of the instrument and device.

There may be two channels on opposed sides of the instrument or device and there are two parts on the other of the instrument or device arranged to be at least partially located in each channel. The two parts may each connect to a single end portion.

The end portion may include a projection (116) arranged, in use, to extend from the opening when the portion is located and held in the opening.

The opening and the or each channel may be in the instrument.

The present invention also includes a surgical instrument when the instrument is as referred to herein.

The present invention also includes a surgical device when the device is as herein referred to.

According to a further aspect of the present invention a method of using an endoscopic surgical instrument and a surgical device comprises holding the instrument and device together by an end portion and a part on one of the instrument or device being at least partially located respectively in an opening at an end region and a channel extending from the opening towards the end of the other of the instrument or device.

The method may comprise rotating the instrument relative to the device.

The method may comprise detaching the device and instrument by pulling a projection (116) on the end portion.

The present invention also includes a method as herein referred to when using an endoscopic surgical instrument or device herein referred to.

The present invention is defined in the claims and elsewhere in this specification.

According to a further aspect of the present invention an endoscopic surgical instrument includes a catcher arranged, in use, to catch a surgical item, the surgical instrument including a movable portion arranged to move from a position that it occupies when the instrument and an item are separate when, in use, a surgical item is being caught.

The movable portion may comprise a resilient portion and may comprise a compressible portion and may include two spaced compressible portions each of which are arranged to move during catching of the item. The resilient portion may comprise at least one flexible jaw which is arranged to flex during catching.

At least part of the flexible jaw may be arranged to contact another portion of the instrument when the instrument and item are separate and which is arranged to move away from that contact during catching. The flexible jaw may be spaced from another portion of the instrument along its length.

The jaw may include a guide arranged to assist in the alignment and commencement of catching. The free end of the jaw may be arranged to at least partially close a gap to cause another gap remote from the jaw to increase and thereby enable catching.

The instrument may include an opening in which a surgical item is arranged to at least partially occupy during catching. The opening may be arranged to be larger than its occupant.

The resilient portion may be arranged to return at least partly towards the position that it occupied when the instrument and item were separate after the item is caught.

According to another aspect of the present invention, a surgical item is arranged, in use, to be caught by a catcher of a surgical instrument is characterised in that:

the surgical item includes a movable portion arranged, in use, to move from a position that it occupies when the instrument and item are separate from each other during catching.

The movable portion comprises a resilient portion. The resilient portion may comprise a compressible portion and may comprise two spaced compressible portions each of which are arranged to move during catching of the item

The resilient portion may be arranged to return at least partly towards the position that it occupies when the instrument and item were separate after the item is caught.

The surgical item may comprise a gastric band including an opening arranged to be caught by the instrument which band may include a hinge adjacent to the opening. The band may include a tab at the end.

According to a further aspect of the present invention, an endoscopic surgical instrument and a surgical item are provided, the instrument including a catcher arranged, in use, to catch the surgical item, at least one of the surgical instrument or the item including a movable portion arranged to move from a position that it occupies when the instrument and item are separate from each other during catching of the item.

The movable portion may comprise a resilient portion and the resilient portion may comprise a compressible portion. Two spaced compressible portions may be provided each of which are arranged to move during catching of the item.

The resilient portion may comprise at least one flexible jaw which is arranged to flex during catching and at least part of the flexible jaw may be arranged to contact another portion of the instrument when the instrument and item are separate and which is arranged to move away from that contact during catching. The flexible jaw may be spaced from another portion of the instrument along its length. The jaw may include a guide arranged to assist in the alignment and commencement of catching. The free end of the jaw may be arranged to at least partially close a gap to cause another gap to be removed from the jaw to increase and thereby enable catching.

The instrument may include an opening which a surgical item being is arranged to at least partially occupy during catching. The opening may be larger than its occupant.

The jaw or jaws may be arranged to flex to allow part of the instrument or item to pass the jaw into the opening.

Catching may be arranged to be effected by at least partially overlapping portions of the instrument and item being relatively moved at least partially away from each other.

Catching may be arranged to be effected by the instrument and item being relatively moved at least partially sideways to each other.

Catching may be arranged to be effected by relative movement of the instrument and item at least partially towards each other.

The resilient portion may be arranged to return at least partly towards the position that it occupies when the instrument and item were separate after the item is caught.

The surgical item may comprise an opening arranged to be caught by the instrument and the band includes a hinge adjacent to the opening but spaced from the instrument when the band is caught. The band may include a tab arranged to extend from the instrument when the band is caught.

The surgical instrument may include a recess arranged to permanently catch the surgical item.

The present invention also includes a method of catching a surgical item to an endoscopic surgical instrument in which at least one of the item or instrument includes a movable portion comprising causing the movable portion to move upon catching.

According to a further aspect of the present invention a gastric band includes a movable portion arranged to move from a position that it occupies when the band is separate from the catcher of a surgical instrument when, in use, an instrument is catching the band.

According to a second aspect of the present invention an endoscopic surgical instrument includes a surgical item catcher, the catcher comprising an opening and a catch member, the catch member being movable between a retracted position in which surgical items can be placed into and removed from the opening and a caught position in which, in use, an item in the opening is prevented from leaving the opening.

The catch member may be movable between the positions from a location remote from the catcher.

The opening may be provided along the length of the instrument and may comprise a channel extending across the instrument. The opening may be located adjacent to the distal end of the instrument. The opening may extend to a depth greater than half the cross sectional area of the instrument at a location adjacent to the opening. The length of the opening may be larger than the width of the instrument adjacent to the opening and may be greater by a factor of at least 2 times the width.

The instrument may be elongate and the catch member may be movable between the caught and retracted positions in the direction of elongate extent of the instrument.

According to a further aspect of the present invention a method of catching a surgical item with an instrument according to the second aspect comprises moving the catch member to the retracted position, locating a surgical item in the opening and then moving the catch member to the caught position.

According to a third aspect of the present invention, an endoscopic surgical instrument comprises a channel at the distal end region in which, in use, the end region of a surgical item is arranged to enter and at least one recessed portion extending from the channel towards the distal end of the channel in which, in use, part of the surgical item is arranged to enter.

Preferably the recessed portion is of sufficient depth such that, in use, when the end region of a surgical item is located in the channel and with a part of the surgical item being located in the recessed portion, the peripheral cross sectional area of the surgical item and the surgical instrument is less than or equal to the peripheral cross sectional area of the surgical instrument towards the proximal end of the instrument adjacent to the channel.

Preferably there are two recessed portions extending from the channel towards the end of the instrument on opposed sides of the instrument. Opposed sides of a surgical item, in use, may be arranged to be located in the opposed recessed portions.

The or each recessed portion may extend to the end of the instrument.

The channel may extend inwardly from the side of the instrument and may be inclined towards the end of the distal end of the instrument at least in the region extending inwardly from the side.

The channel may extend along an elongate extent of the instrument along at least part of its length. The channel may extend along the elongate extent of instrument and may be inclined with respect to the elongate extent along at least part of its length. Part of the length of the channel may extend inwardly from the side and another part of the length of the channel may extend along an elongate extent of instrument and may be inclined along that elongate extent towards the side of the channel from which the channel extends inwardly.

The present invention includes a surgical instrument in combination with a surgical item in which part of the surgical item is located in the channel and part of the item is located in the or each recessed portion.

The surgical item may comprise a gastric band. The gastric band may include a loop at the end region. The gastric band may be self supporting at the end region. The gastric band may end in a tab. The tab may be arranged to end towards the external entrance to the channel when part of the band is in the channel. The tab may be arranged to be located wholly within the channel when part of the band is in the channel or may be arranged to extend beyond the channel. The tab may be arranged to extend in the channel at the same angle as the channel extends inwardly from the side of the instrument.

The present invention includes a gastric band for use with the third aspect of the present invention.

According to another aspect of the present invention, a method of using an endoscopic surgical instrument comprises connecting a surgical item with the instrument with the cross sectional periphery of the connected instrument and item being less than or equal to the adjacent cross sectional periphery of the instrument.

Alternatively or additionally the method may comprise using a surgical instrument by locating part of a surgical item in a channel at the distal end region of the instrument with part of the item extending in a recessed portion of the instrument which recessed portion extends from the channel towards the distal end of the instrument.

The method may comprise catching the surgical item as described and subsequently pulling the surgical instrument back together with the surgical item. The method may comprise initially pushing the surgical item forwards, then catching the surgical item and then pulling the instrument back together with the surgical item.

The method may comprise using a surgical instrument according to the third aspect of the present invention.

7

According to a fourth aspect of the present invention, an endoscopic surgical instrument includes a recess arranged, in use, to permanently catch a surgical item.

The recess may include at least one inwardly extending lip arranged, in use, to allow a surgical item to enter the recess but permit withdrawal of the item from the recess. Alternatively or additionally the recess may taper inwardly from the entrance to the opening.

The recess may be formed in the end of the instrument and may extend towards the proximal end of the instrument.

The instrument may include a hinge adjacent to the recess permitting the recess to move relative to another portion of the instrument. The hinge may be formed by a narrow portion of the instrument. The hinge may permit movement in two opposed directions. Movement about the hinge may be effected by external forces being applied to the distal end region of the instrument.

The hinge and the portion defining the recess may be integrally formed.

The present invention includes a surgical item in combination with the surgical instrument. The surgical item may include at least one projection arranged, in use, to allow the item to enter the recess and to cooperate with the instrument to prevent withdrawal of the item from the instrument. The item may include a barbed tip. The item may include at least one surface that diverges outwardly in a direction away from the end of the item and that diverging surface may end in a portion that extends inwardly from the surface. The peripheral cross section of the item and instrument may be less than or equal to the peripheral cross section of an adjacent part of the instrument.

The present invention also includes a surgical item for use with a surgical instrument in accordance with the fourth aspect.

According to another aspect of the present invention, a method of using a surgical instrument comprises permanently catching a surgical item in a recess of the instrument. The method may comprise a one way cooperating mechanism on the item and instrument preventing disconnection. The method may comprise pushing a barb on the item past an inwardly projecting lip of the recess.

The method may comprise pushing the item into the end of the instrument.

The method may comprise pulling the instrument after catching the item.

The method may comprise dividing the item such as for example by cutting the item after the item has been connected to the instrument.

According to a fifth aspect of the present invention an endoscopic surgical instrument and a surgical item include cooperating formations at end regions whereby relative movement between the item and the instrument at those end regions is arranged to cause engagement between the item and the instrument.

The instrument may be elongate and part of the relative movement between the formations may be arranged to be transverse to the elongate extent. Part of the movement may also be arranged to be in the direction of elongate extent.

When the instrument and item are engaged relative movement may be prevented in a direction transverse to the longitudinal axis. Alternatively or additionally relative movement may be permitted to a limited extent in the direction of elongate extent of the instrument or may be prevented in the direction of elongate extent

The instrument may include a channel transverse to the elongate extent and a recessed portion extending from the channel towards the end of the instrument. The recessed portion may extend towards the centre of the cross section of the instrument or to the centre or beyond the centre. The channel may extend from one side to the other of the instrument and may extend towards the centre or to the centre or beyond the centre of the instrument. The item may include an elongate portion arranged to be located in the recessed portion and to be parallel to or aligned with the elongate axis of the instrument when the item and instrument are engaged.

The end of the item may include a cross portion arranged to abut with the instrument to restrict relative movement in the direction of elongate extent of the instrument when the parts are connected. The cross portion may be an elongate portion such as a cross T or a part spherical shape.

The item and the instrument may be a loose fit or a tight fit or a friction fit when they are engaged.

The combined periphery of the item and instrument, when connected, may be less than or equal to the periphery of the instrument adjacent to the connection.

The present invention includes an endoscopic instrument for use with the instrument and item in accordance with the fifth aspect.

According to another aspect of the present invention a method of connecting an endoscopic surgical instrument and a surgical item comprises causing cooperating formations at their end regions to move relative to each other to cause engagement between the item and instrument.

The present invention also includes a method of connecting an endoscopic surgical instrument and a surgical item in accordance with the fifth aspect.

According to a sixth aspect of the present invention, a surgical item includes a tab at the end, the tab being arranged, in use, to be gripped by an endoscopic instrument.

The tab may be arranged to be gripped by an endoscopic instrument to assist in the item being caught by or released from or both of a further endoscopic instrument.

The tab may comprise a formation. The tab may be integrally formed with the rigid item.

The tab may extend at an angle to the end region of the item adjacent to the tab. The tab may be arranged, in use, to extend outwardly towards or to or beyond the side of an opening of an endoscopic surgical instrument to which it is able to be connected to.

The tab may extend from the end of an opening such as a loop in the item.

The present invention also includes an endoscopic instrument and the item in accordance with the sixth aspect, the instrument including a channel at an end region extending inwardly from the side of the instrument with the tab being arranged to be located at least partially in the channel when the instrument and item are connected. The channel may extend inwardly and forwardly towards the end of the instrument and the tab may extend at the same angle.

The present invention also includes a method of manipulating a surgical item by providing a tab at the end of the item and gripping the tab with an endoscopic surgical instrument and subsequently manipulating the item.

The present invention also includes a method of manipulating a surgical item when the item is in accordance with the sixth aspect.

According to a seventh aspect of the present invention a gastric band includes an end portion arranged, in use, to be manoeuvred by an endoscopic instrument and a hinge integral with the band adjacent to the end of the end of the band about which the band can pivot.

The hinge may be arranged to permit pivoting about one axis but to inhibit pivoting at that region about any other axis.

The hinge may comprise a region of reduced thickness. The reduced thickness may extend from one side to the other.

The band, in the region of the hinge, may be thicker in the direction of extent of the hinge than in a direction perpendicular to the extent of the hinge.

The band may include an opening such as a loop at the distal end of the band. The opening may be arranged, in use, to be engaged by an endoscopic surgical instrument.

The present invention also includes a method of manoeuvring a gastric band comprising causing the band to move about a hinge that is integrally formed with the band.

The present invention also includes a method of manoeuvring a gastric band when the band is in accordance with the seventh aspect.

According to an eighth aspect of the present invention an endoscopic surgical instrument includes a catcher at one end region for a surgical item, and a hinge about which the catcher can move, movement of the catcher in at least one direction being arranged to be effected by applying a force to at least one arm on the other side of the hinge.

The hinge, arm and catcher may be integrally formed.

The arm may be opposed to another part of the instrument but spaced therefrom when no force is applied to the arm and may be caused to move closer to the opposed part when a force is applied thereto.

The arm may be located towards the distal end with the catcher being located more towards the distal end.

The arm may extend to the end of the instrument.

When no force is applied to the arm the catcher may be in a position in which a surgical item is caught.

The catcher may be arranged to form part of an enclosure for a surgical item. The catcher may be arranged to afford access to the enclosure when moved about the hinge and to restrict access when not so moved.

The catcher may be arranged, in use, to catch an end region of a gastric band.

The present invention also includes a method of operating an endoscopic surgical instrument comprising moving a catcher at an end region on one side of a hinge about the hinge in at least one direction by applying a force to an arm on the other side of the hinge.

The present invention includes a method of operating an endoscopic surgical instrument when the instrument is in accordance with the eighth aspect of the invention.

According to a ninth aspect of the present invention a gastric band includes an end region arranged, in use, to be connected to a surgical instrument, the end region including an opening spaced from the end with the end of the band being arranged to pass around part of a surgical instrument and to be passed back through the opening to form a loop.

The parts of the band that define the loop may be arranged, in use, to be connected to each other.

The part of the band that, in use, is located in the opening may be restrained from passing through the opening by friction between the parts of the band.

The end region of the band may be in the shape of a tape and the tape may be arranged to undergo at least a partial twist when forming the loop.

The loop may be arranged, in use, to pass through an opening in an endoscopic surgical instrument.

The present invention includes an endoscopic instrument in combination with a gastric band.

According to a further aspect of the present invention a method of attaching the end region of a gastric band to an endoscopic surgical instrument comprises forming a loop that attaches to the instrument with the loop being formed by passing the end of the band through an opening in the bend.

The present invention also includes a method of attaching the end region of a gastric 5 band to an endoscopic surgical instrument when the band is in accordance with the ninth aspect.

According to a tenth aspect of the present invention a gastric band includes an opening in an end region, the opening being arranged, in use, to be engaged by an endoscopic surgical instrument which is able to manoeuvre the band.

The band may be in the form of a tape at the end region and the opening may extend along the band in the elongate extent of the band.

According to a further aspect of the present invention, a method of manoeuvring a gastric band comprises inserting an arm of an endoscopic surgical instrument into an opening in the end region of the instrument and moving the arm to move the band.

The opening may be elongate along the elongate extent of the band.

The method may comprise the arm engaging the opening to move the band in at least one and preferably in opposed directions of elongate extent of the band.

The method may comprise moving the band in directions transverse to the extent of the opening.

The method may comprise gripping the band with a jaw of the instrument that clamps the band between the jaw and the arm and then manoeuvring the end region of the band.

The present invention also includes a method of manoeuvring a band when the band is in accordance with the tenth aspect.

Any of the aspects referred to herein may be combined with any of the other aspects.

The present invention may be carried into practice in various ways but several embodiments will now be described by way of examples with reference to the accompanying drawings, in which:

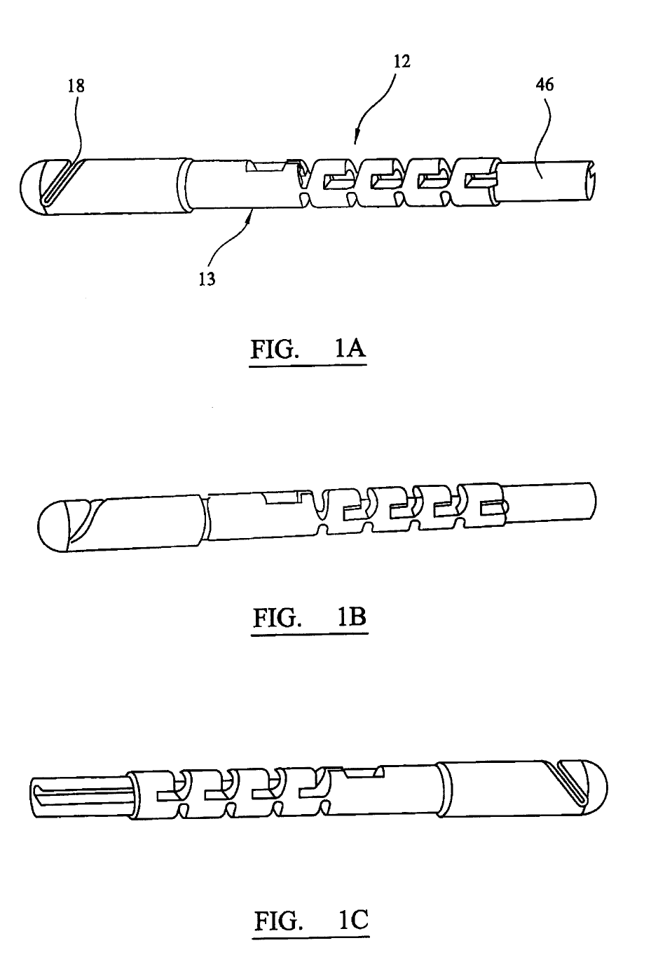

FIGS. 1A, 1B and 1C show a distal end of an endoscopic surgical instrument from various angles;

FIGS. 2A, 2B and 2 C show in sequence the snaring of a gastric band or suture;

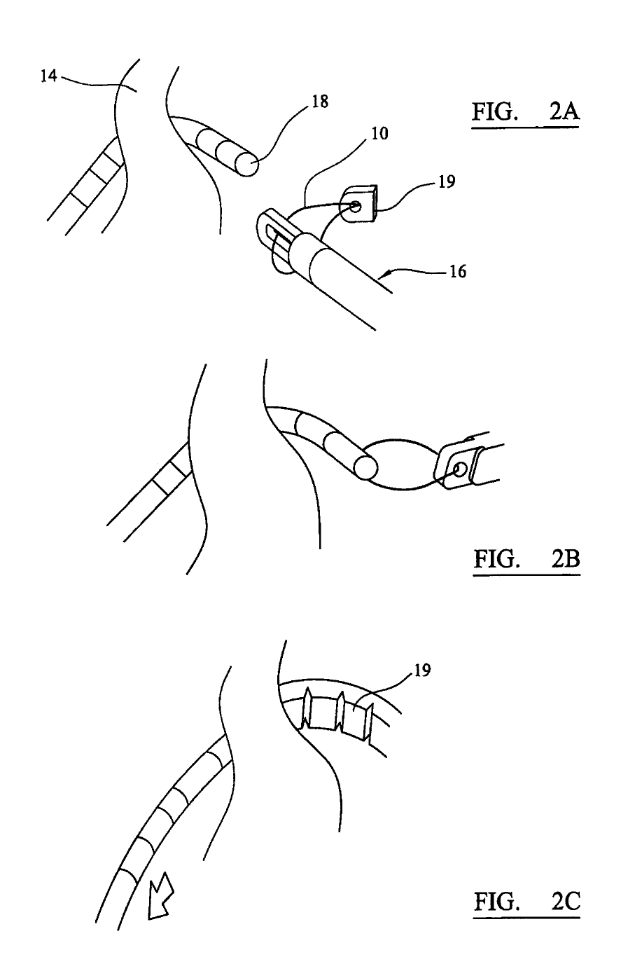

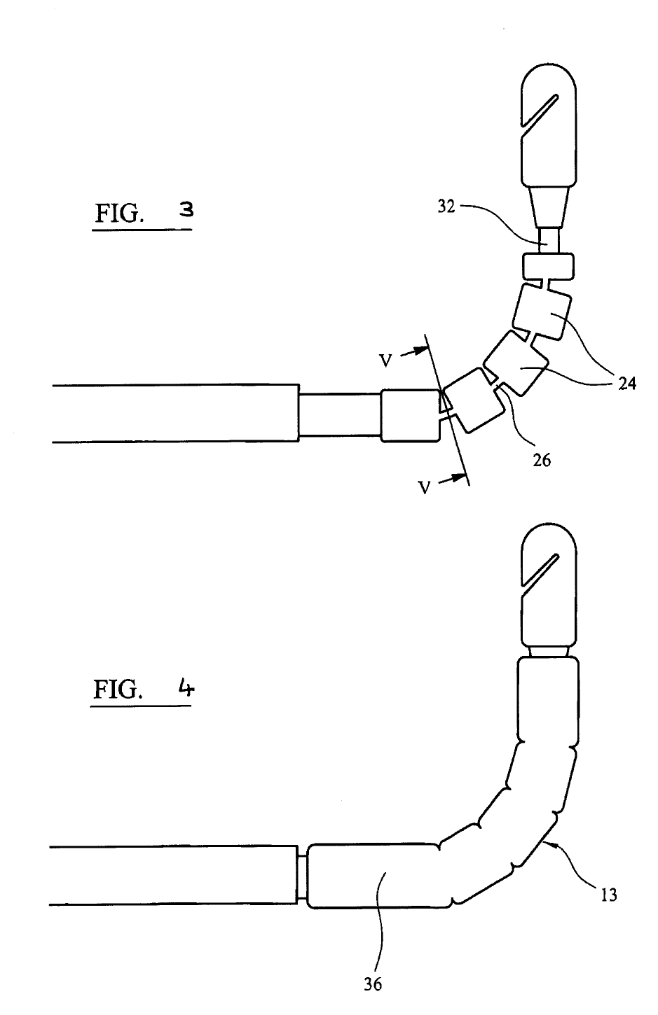

FIGS. 3 and 4 are side views of the distal end of the instrument with an articulated section being exposed in FIG. 3;

FIG. 5 is an enlarged sectional view along the line v-v of FIG. 3;

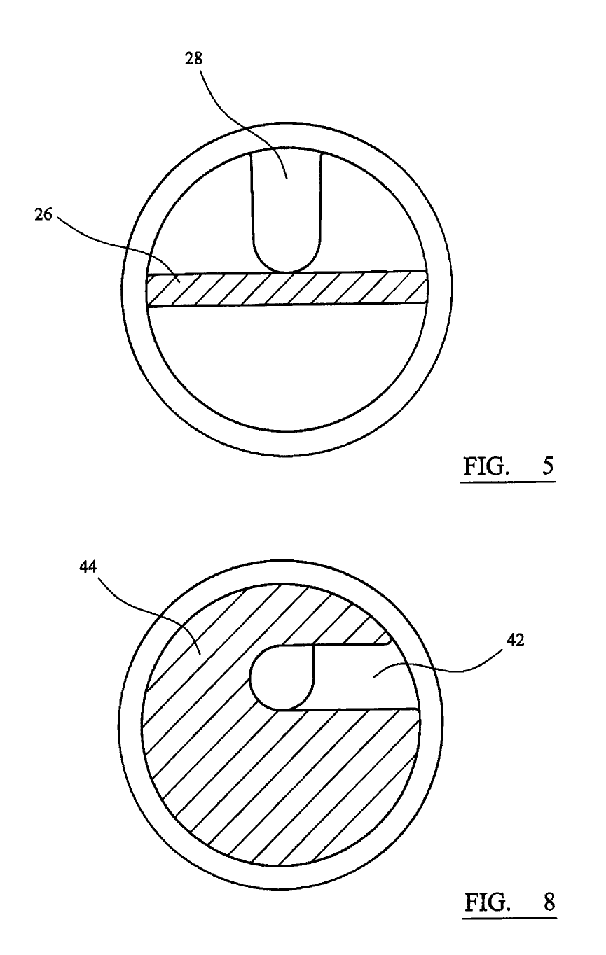

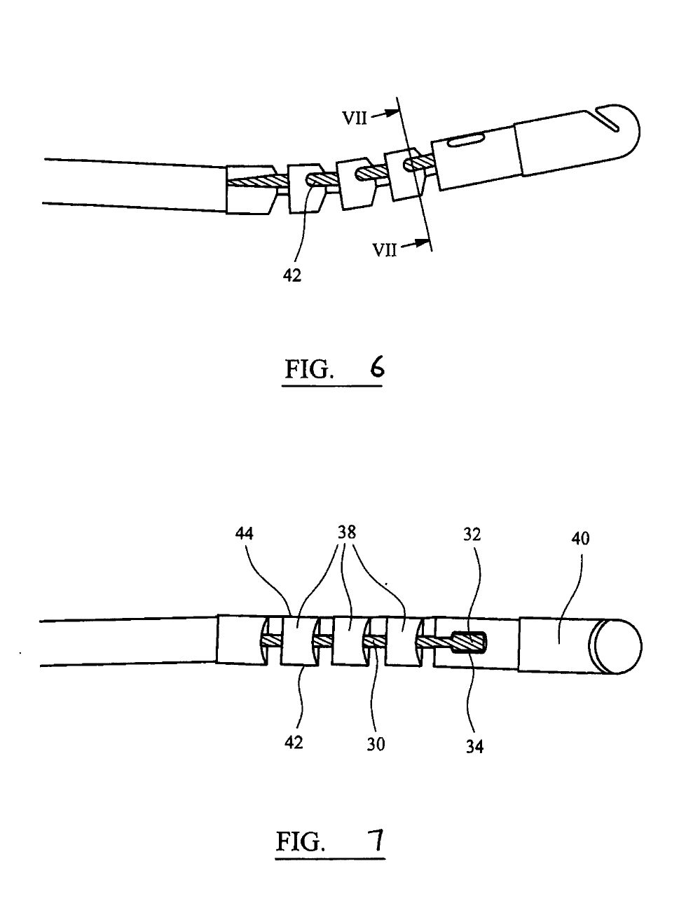

FIGS. 6 and 7 are side and plan views respectively of a distal part of the instrument according to an alternative embodiment;

FIG. 8 is a sectional view along the line viii-viii of FIG. 6

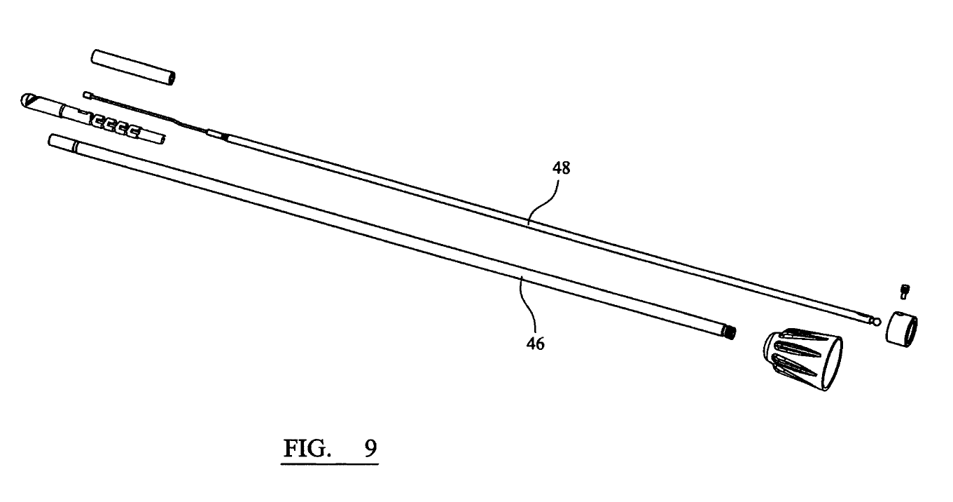

FIG. 9 is an exploded view of the instrument, without the control that can cause the segments to be articulated;

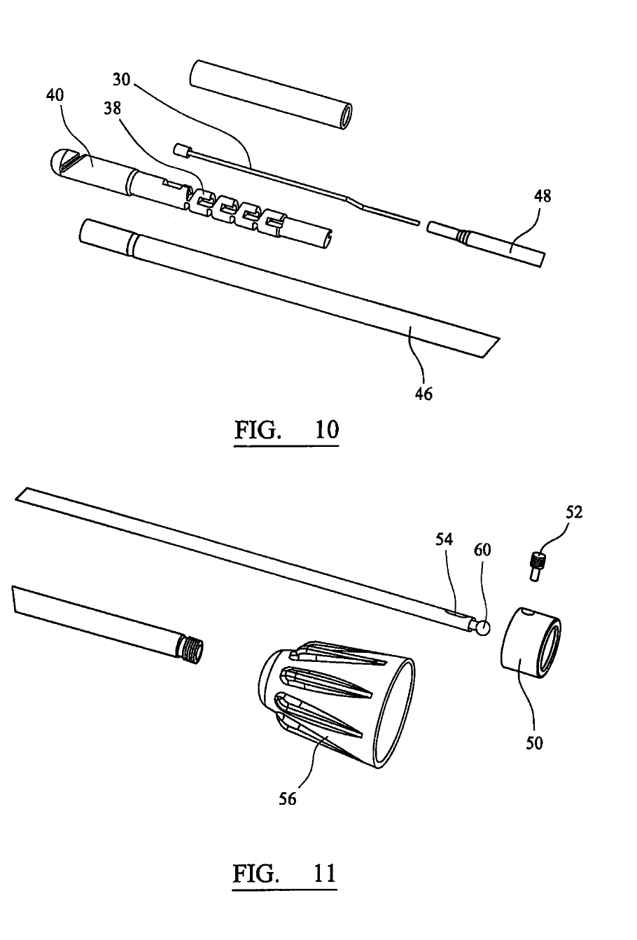

FIG. 10 is an enlarged view of the distal end of FIG. 9;

FIG. 11 is an enlarged view of the proximal end of FIG. 9;

FIGS. 12 and 13 are cross sectional views through the assembled proximal end of the instrument, and

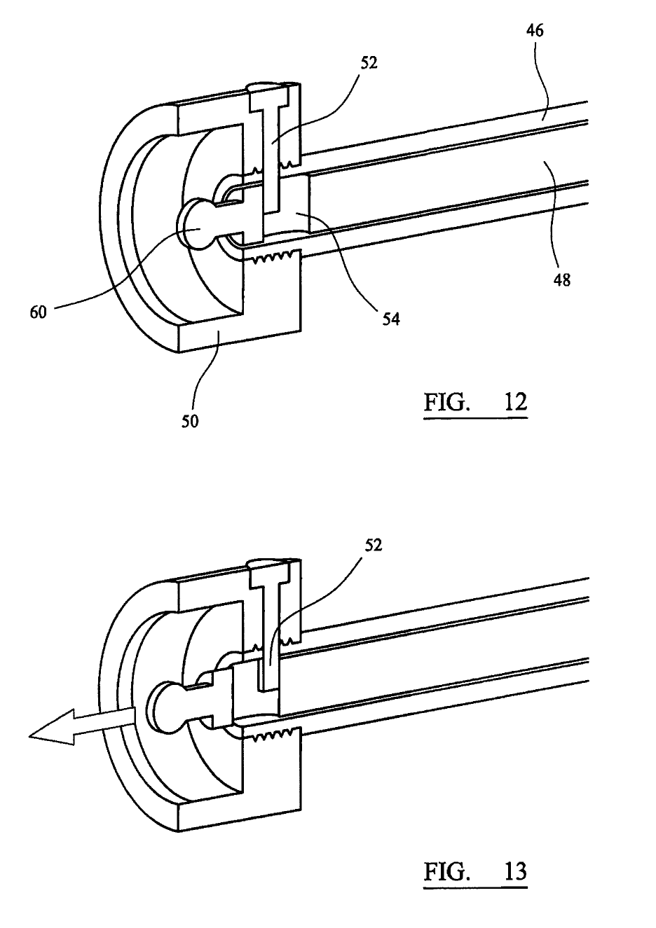

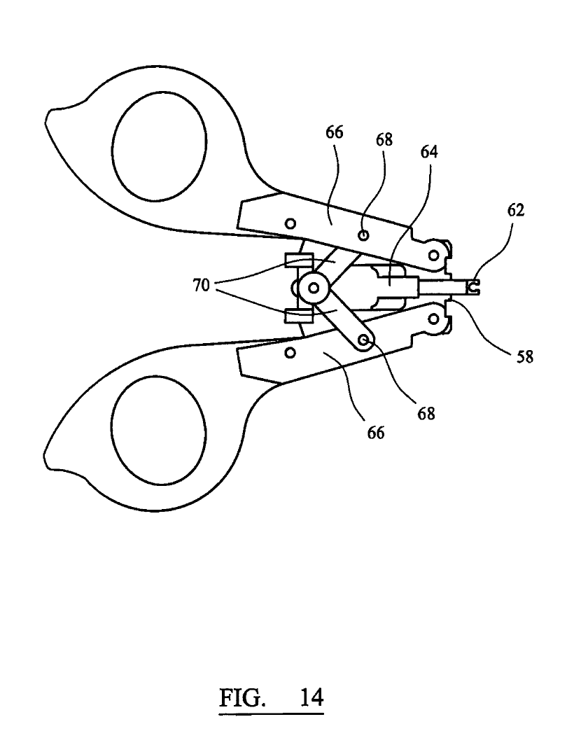

FIG. 14 is a top view of a control mechanism to cause the articulation of the instrument,

FIGS. 15 to 21 and 24, 28 and 29 are views showing different cooperating features of 30 the end region of the gastric band and the catcher at the end of the endoscopic instrument,

FIGS. 22, 23 and 25 to 27 are views of different forms of catchers;

FIG. 30 is a view of the end region of a gastric band, and

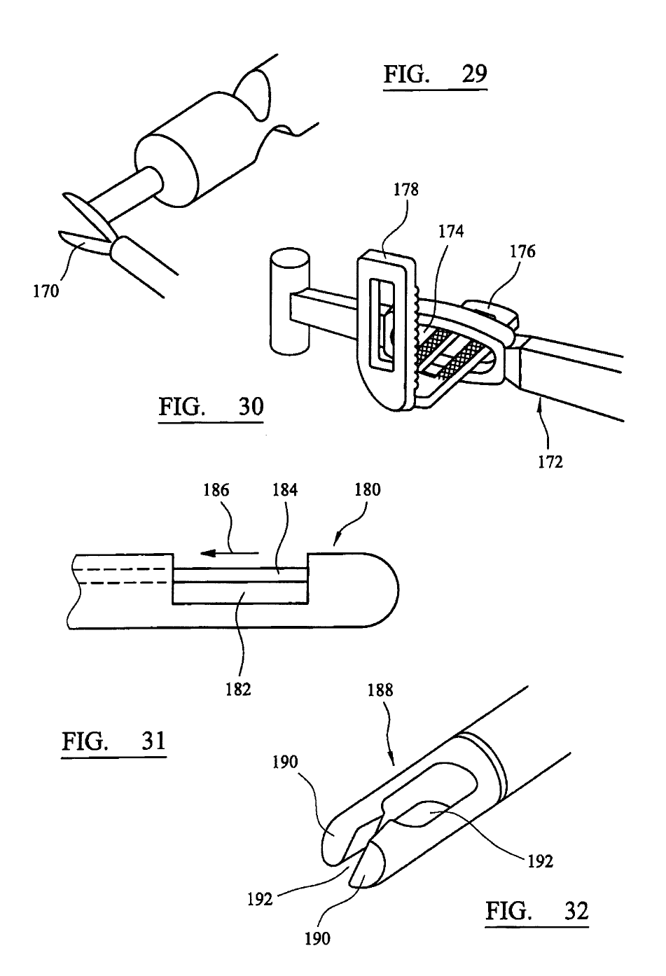

FIGS. 31 and 32 are views of further forms of catchers.

FIGS. 1A, 1B and 1C show a distal end of an endoscopic surgical instrument 12. The distal end comprises an articulated section 13 formed from a single piece moulding. The articulate section 13 comprises a series of sections 24 that can be caused to move towards each other when the instrument is activated such that the articulated section 13 forms a curve or shape rather than being substantially elongate when unactivated. A catcher 18 is formed at the end of the articulated section 13. An end 46 of the moulding is of reduced external diameter in order to fit within a tube 46 of another part of the instrument

FIGS. 2A, 2B and 2C show the sequence of snaring a suture 10. The endoscopic instrument 12 is inserted through the body tissue and is then articulated behind a section of stomach 14. A further instrument 16 then offers up the suture 10 in the form of a loop to the catcher 18 of the instrument 12. Once caught, as shown in FIG. 2B, the curve on the end of the instrument can be reduced and the instrument 10 can be retracted, possibly at the same time, to bring the suture 16 back which suture, in turn, brings the gastric band 19 partially around the stomach 14.

As shown in FIG. 3 the articulated section of the distal end is made up of a series of sections 24. The sections 24 and the catcher 18 are integrally moulded such as by injection moulding polymer, such as polypropylene. Each section 24 has a circular periphery.

Each section is connected to an adjacent section by a hinge 26 that extends across the centre of the section from one side to the other. Each hinge has the same angular relationship to the elongate extent of the instrument.

The sections each include an opening 28 through which an articulation operator 30 comprising a stainless steel micro cable can extend. The end of the operator 30 includes an enlarged head 32 comprising a crimped end of the cable that sits in a recess 34 located at the end of the most distal segment.

In order to articulate the distal end, tension is applied to the operator 30 which pulls the sections to the position shown. It will be appreciated that by varying the tension of the operator the length of the sections along one side is varied. Accordingly the sections in this or other embodiments can occupy any curvature. The sections in this or other embodiment may also articulate in the opposite direction by pushing the operator 30 to increase the length of the sections on that side such that the gaps between the sections on the other side are reduced. The hinges 26 may assist in bringing the sections back to a predetermined position as a result of the natural flexure of the hinge.

Whilst integrally formed sections are caused to articulate it will be appreciated that any form of articulation may be used to cause the end region of the endoscope to alter its orientation.

When the sections are activated to the desired limit position, a restriction means to stop the sections from moving further is required. In the prior art, the restriction means comprises the adjacent faces of the section abutting. Whilst this is a possibility it is advantageous that they do not abut. That is because, when catching a suture, a small amount of flexure may be desirable. That flexure may be caused by tension in the suture being exerted as the suture slides along the instrument, which tension is released when the suture enters the opening 20 of the catcher. Moreover, if the sections avoid abutment the risk of cracking/breaking up of the sections is reduced. Accordingly, the sections are delimited before they abut by limiting the tension that can be applied to the control member 30 as herein described.

FIG. 4 shows the articulated section 13 of the instrument, excluding the catcher, enveloped by a heat shrunk sleeve 36. The sleeve 36 prevents any parts that may come loose from leaving the instrument. The sleeve 36 prevents tissue and the suture from being caught in the gaps between the sections. Accordingly, the sleeve prevents tissue from becoming trapped in the instrument. The sleeve 36 may be used with the embodiment disclosed in FIGS. 6 and 7.

In FIGS. 6 and 7, the sections 38 and the catcher 40 are also integrally moulded such as by injection moulding polymer. The only difference between FIGS. 6 and 7 and the previously described embodiment is in the form of the sections 38. As shown in FIGS. 6, 7 and 8, each section includes a slot 42 along one side for half the length of the section and a hollow section along the other half in order to locate the operator 30. This arrangement facilitates injection moulding of the articulated section.

Referring now to FIGS. 9 to 11 these show, in enlarged view, the details of the sections 38 and catcher 40 together with the control 30 and how these parts are connected to the remainder of the instrument.

The trailing section 38 abuts a hollow member comprising the tube 46. The control 30 comprising the cable is attached to the distal end of a rod 48 that is located within the tube and is able to slide along the tube.

As shown in FIGS. 12 and 13, a collar 50 surrounds the end region of the tube 46 and is fast therewith. The collar has a detent 52 that passes through the collar and the tube 46 and extends into a slot 54 of the rod 48. Relative axial movement of the rod to the tube is restricted by abutment of either end of the slot 54 with the detent 52. In this way curvature of the sections in either direction is also restricted, and it will be appreciated that this restriction prevents the sections from abutting as previously mentioned.

Referring back to FIGS. 10 and 11, a cone 56 is slidably mounted on the tube 46. The sliding movement in the proximal direction is restricted by abutment with the collar. An internally threaded portion (not shown) of the cone 56 extends beyond the end of the tube and the collar whereby an external threaded portion 58 of the control mechanism shown in FIG. 14 can be attached to the instrument.

The proximal end of the control rod 48 includes a reduced section leading to an enlarged head 60. The head 60 is located in a seat 62 of the control mechanism such that the rod 48 is constrained to move in the distal and proximal direction with a slider 64 in which the seat 62 is formed.

As shown in FIG. 14, in order to vary the curvature of the sections a user moves two handles 66 about their respective pivots 68. That movement causes links 70 connected respectively to each handle at pivots 69 and the slider 64 to pivot about those connections to retract or extend the slider and thereby the rod 64.

FIG. 15 shows the catcher 72 having a groove 74 extending alone one side of the end region from the end of the instrument. The groove 74 leads to a groove 76 that is transverse to the groove 74. The end of the gastric band has an enlarged end 78 in the form of a T. The enlarged end is formed on the end of a flexible rod 80.

In order to trap the end of the band the rod 80 is caused to lie within the groove 74 with the enlarged end being in the transverse groove 76. The cooperation between the catcher and the end region of the band may be a loose fit or may be a friction fit. The end region of the band may be caught by manipulation and alignment with the catcher. Alternatively a further endoscopic instrument (not shown) may be used to cause the end region to be caught as may be the case with any of the embodiments. The fit of the enlarged end in one or both grooves may be a loose fit or a tight fit or a friction fit.

The rod 80 and the end 78 preferably do not extend beyond the side of the endoscopic instrument. Accordingly the endoscopic instrument can be pushed through the tissue of the body to make the catcher 72 available for the gastric band. Then the band can be caught and pulled back through the tissue without snagging on the tissue.

FIG. 16 is similar to FIG. 15 in construction and operation and may have any of the features of FIG. 15. Accordingly only the differences will be described. In FIG. 16 the enlarged end 82 of a gastric band is in the form of a sphere. The sphere and/or the rod 80 may be a friction fit, a tight fit or a loose fit. The instrument may be able to rotate about the sphere or visa versa about the longitudinal extent of the rod.

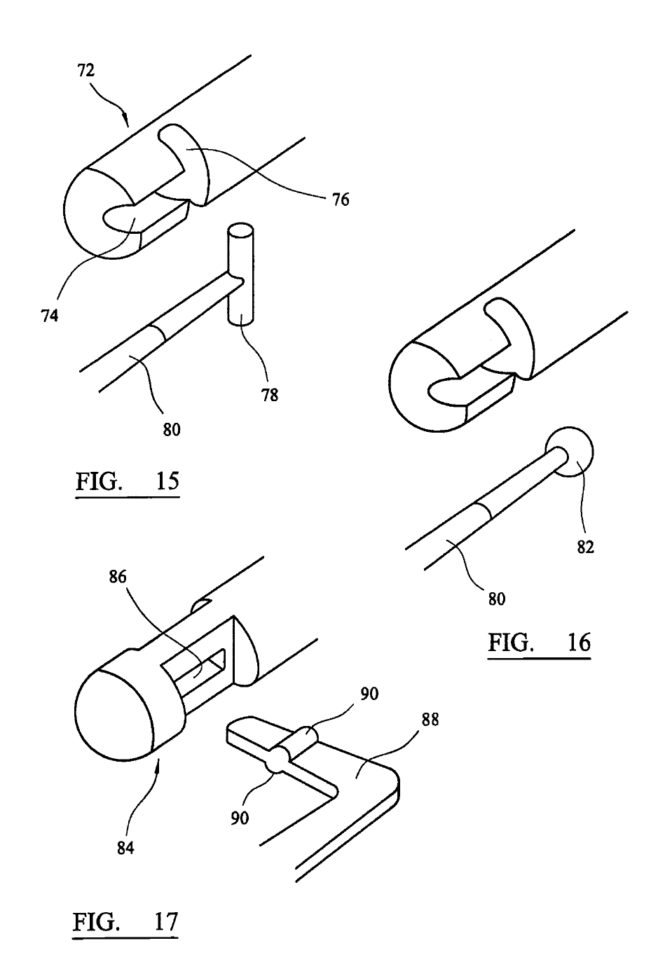

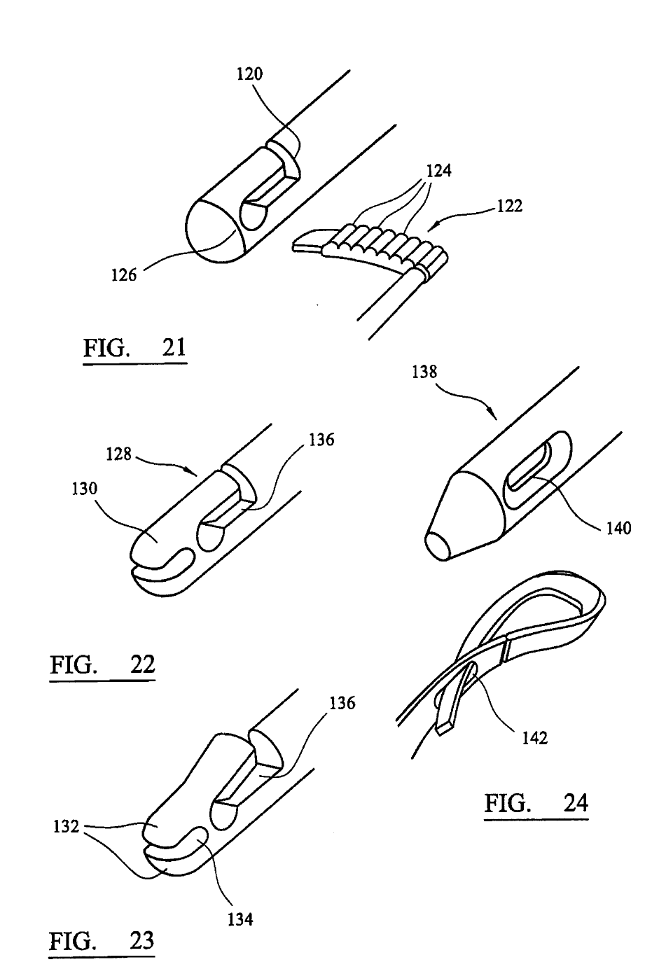

FIG. 17 shows the catcher 84 comprising a slot 86 formed in a region of reduced thickness. The end region of the gastric band has a turn 88 that extends transversely to the elongate extent of the end region of the instrument. Although not shown, the end of the instrument may include a channel similar to the channel 24 shown in FIG. 15 within which at least part of the band is located, as may the instruments shown in FIG. 21 or 24.

To attach the band to the instrument the turn 88 is caused to pass partially through the opening with a flexible portion 90 comprising two ribs on either side of the turn 88 being caused to be compressed or flexed to enter the slot 86 or alternatively to be compressed and flexed as the turn passes through the slot and then to pass beyond the slot 86 and then flex or decompress outwardly again. The end of the turn 88 is tapered inwardly towards the end to assist in the alignment. The end of the turn 88 preferably does not extend beyond the far side of the instrument when the band is connected.

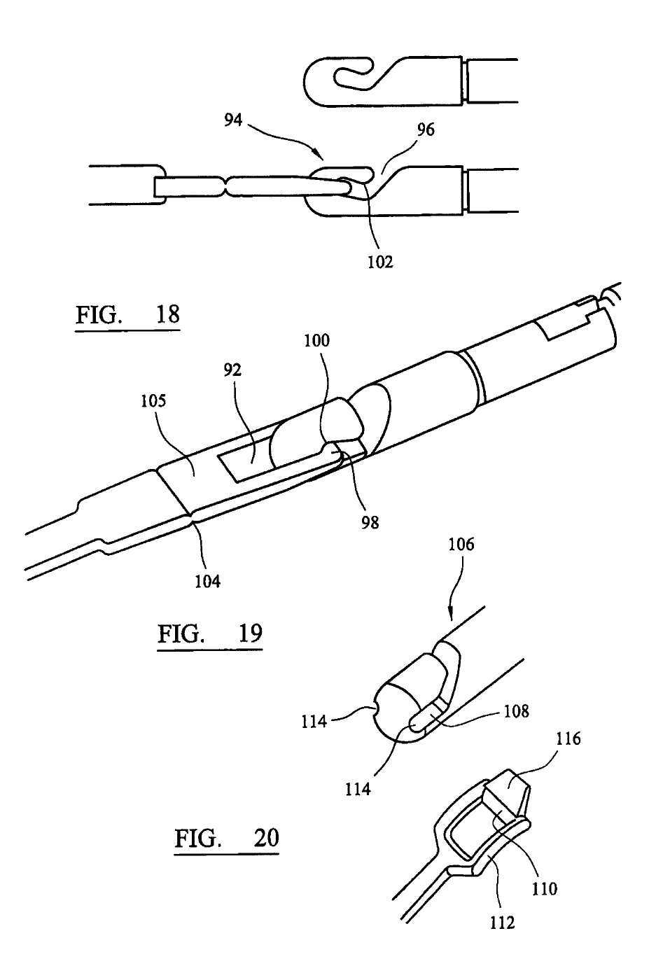

FIGS. 18 and 19 show the end region of the gastric band having an opening 92. The catcher 94 has a channel opening 96 through which the end wall 98 of the band can pass to abut the end 100 of the channel. The band can then be pulled by the instrument. Towards the closed end of the channel the channel is inclined upwardly towards the side of the instrument having the opening 96 to the channel to assist in preventing inadvertent detachment of the band from the channel.

In order for the wall 98 to pass from the opening 96 to the end of the channel the wall must squeeze past a flexible portion 102 which is compressed or flexed or squeezed as the side wall passes and which then flexes or decompresses back after the wall 98 has passed. Such an arrangement allows not only for the band to be pulled but also permits the instrument to be manoeuvred such that a force that may cause the band to leave the catcher is resisted by the flexible portion.

The band includes a region of reduced thickness that forms a hinge 104. This allows the band to bend and easily follow the instrument as the band is being pulled or manoeuvred and may also assist in the attachment of the band to the catcher. The band has a flat region 105 to assist in gripping the band.

FIG. 20 shows the catcher 106 comprising a channel 108 with the end wall 110 of a loop of the band being able to enter that channel and to then be able to be pulled or manoeuvred by the instrument as described in relation to the embodiment of FIG. 19. A flexible portion 102 may or may not be present in the channel.

Two opposed side walls 112 of the loop lie within grooves 114 that extend from the channel 108 to the end of the instrument. The side walls 112 may lie completely within the grooves such that the band can easily be pulled back through an opening. The side walls 112 may be 1 mm in the direction across the opening and 0.5 mm deep. The cooperation of the side walls with the grooves may assist the instrument being able to twist the band as the end of the band is fixed against rotation with respect to the end region of the band.

The band includes a formation comprising a tab 116 that extends upwardly and outwardly at generally the same angle as that of the entry to the channel 108 and may or may not protrude beyond the instrument when the end wall 110 is at the end of the channel. The tab may be able to be gripped by a further endoscopic instrument (not shown) to assist in locating the end wall in the channel and possibly also to enable the band to be pulled out of the channel.

FIG. 21 is similar to the embodiment described in FIG. 17. The catcher 118 has a channel 120 and a bend 122 on the end of the band. The bend 122 is inserted from the side into the channel. The bend includes a flexible portion 124 comprising a plurality of ridges that can be compressed or flexed as the band is inserted into the channel. Alternatively or additionally the free end of the channel may flex about a hinge 126 to increase the gap afforded by the channel as the bend is forced through.

The bend 122 may or may not extend beyond the far side of the catcher.

FIGS. 22 and 23 are alternative ways of assisting a catcher 128 to flex. Again a bend (not shown) is inserted from the side such as the bend 122 of FIG. 19 or the bend 88 of FIG. 15. The insertion of the bend may effect the pivoting about a hinge 130. Alternatively or additionally an endoscopic instrument (not shown) may be used to push end flanges 132 separated by a split 134 towards each other or to squeeze the flanges towards each other to increase the size of the channel 136.

FIG. 24 shows the catcher 138 comprising a slot 140. The end region of the band is in the form of a tape with an opening 142 being formed in the tape, spaced from the end.

In use, the tape is threaded through the slot 140 and is then pushed back through the opening 142 in the tape. The opening 142 is elongate in the elongate direction of the tape and the tape may undergo at least a partial twist between the opening 142 and the end of the tape.

Friction at the opening 142 may assist in holding the band to the instrument. Alternatively or additionally the overlapping parts of the tape may be fastened together.

A further endoscopic instrument (not shown) may be involved in threading the tape through the slot 140 and/or the opening 142 and/or fastening the caught tape.

FIGS. 25, 26 and 27 are views of catchers 142, 144 and 146 in the form of arms that can have their free ends flexed to allow a loop of a band to pass but which can flex back to trap the band in an opening 148.

In FIG. 25 the free end of the arm extends away from the end of the instrument. The end of a band is located in a recess 150 of the instrument facing the free end of the arm. When the instrument is pulled away from the band the band will rise up a ramp 152 to push against the overhanging tip of the arm to flex the arm about a hinge 154. The band then enters the opening 148 and the arm flexes back.

In the relaxed positions of the arms shown in FIGS. 25 to 27 the free end of the arm of the catcher may abut another part of the catcher or may be spaced therefrom.

In FIG. 26 the forward facing end of the catcher 146 includes an upwardly and rearwardly facing ramp 156 that a band is urged up when the end of the instrument is urged towards a loop in the band. The overhanging tip of the arm is then flexed about a hinge 158 to allow the band to move into the opening 148. The arm then flexes back.

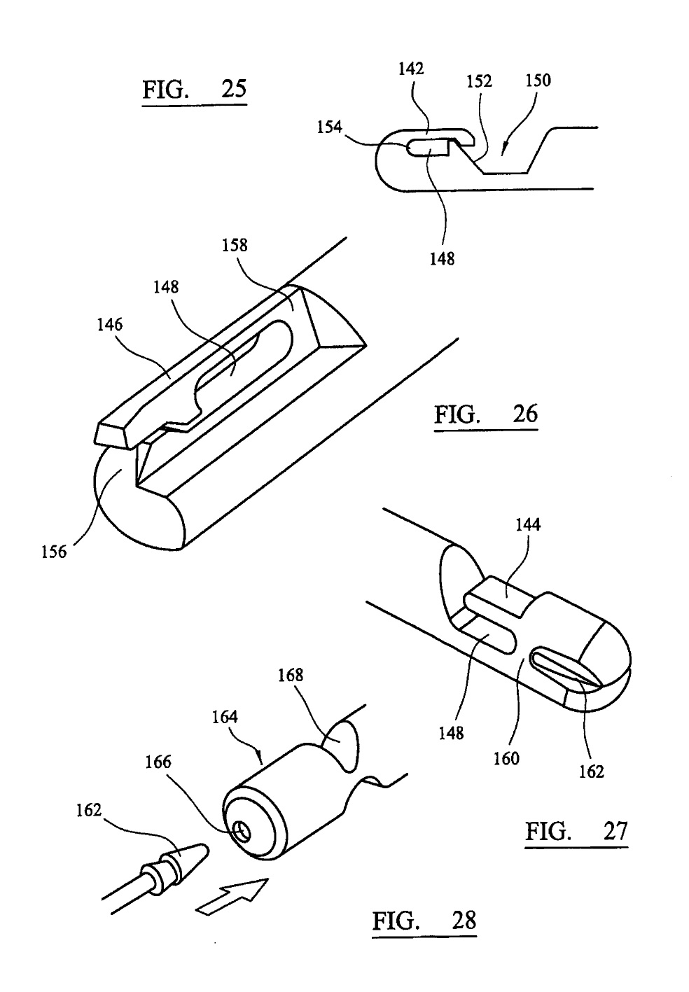

FIG. 27 has a rearwardly facing arm 144 which is able to capture a band by pulling the band in a similar manner to that described in FIG. 23. In this embodiment though a downwardly and forwardly facing ramp is formed on the free end of the ramp to guide the band down and/or to flex the arm about the hinge 160. Again the arm will flex back when the band enters the recess.

The hinge may be assisted in its operation by the free end of the instrument having an opening 162 extending inwardly towards the hinge. One side of the opening may be pushed towards the other or both sides may be urged towards each other to assist in opening the arm as described in relation to FIGS. 22 and 23.

Alternatively or additionally the opening 162 may serve as a holder for the band. For instance the free ends that define the opening 162 may be flexed apart to allow the band to enter an opening spaced from the free ends with the free ends then snapping back. The opening of the free ends may be assisted by pushing the arm 144 towards the opening 148 to cause the free ends to move about the hinge 160.

In each of the embodiments of FIGS. 15 to 27 there is the option of detaching the caught band. For instance if a force that is the reverse of that which caused the capture is applied then detachment will occur. Alternatively or additionally a further endoscopic instrument may be used to apply the reverse force to that which caused capture or to manipulate part of the catcher to effect release or both.

FIGS. 28 and 29 show the band having a barbed tip 162. The holder 164 includes a forwardly facing socket 166 that the tip 162 enters to hold the tip. The socket 166 may comprise a cylindrical or inwardly tapering wall or alternatively or additionally may have an inwardly extending lip or lips that assist in the retention of the tip 162.

The instrument includes a narrow portion 168 adjacent to the socket that may provide a hinge for the holder.

To detach the band from the holder the band is cut by a pair of scissors 170 adjacent to the socket.

FIG. 30 shows the end region of a band 172 having a T end as shown in FIG. 13. It will be appreciated that the end of the band may have any formation as herein described or any other formation that is able to be caught by the holders. The band includes an opening 174 into which a manipulating member 176 in the form of a jaw can be inserted. A further manipulating member 178 in the form of a jaw may be pivotally connected to the other jaw whereby one side of the band that defines the opening can be gripped between the jaws. The jaws are provided on a different endoscopic instrument to that with the holder and the jaws can be opened and closed from outside of the body cavity in a well known manner.

The gripped band can then be moved into or out of engagement with the holder. The manipulation is further assisted in that the jaw within the opening can urge the band in the direction of its elongate extent by abutting with the ends of the opening 174 even if the grip of the jaws is not that strong or indeed even if there is no such grip.

The catcher 180 shown in FIG. 31 has an opening 182. A rod 184 closes the opening to catch a band (not shown) in the opening. The rod can be retracted in the direction of arrow 186 to allow a bands loop to be placed in or removed from the opening 182. The rod 184 can be moved to open or close the opening by being pulled or pushed from outside the body of a patient.

FIG. 32 shows an embodiment similar to FIGS. 22 and 23. In FIG. 32 though the catcher 188 has a pair of jaws 190 at the end which are able to be flexed away from each other and then to spring back to trap a band in the opening 192. The jaws 190 may contact each other in the position shown and may be biased towards each other to urge the jaws into contact. Alternatively the jaws may be spaced from each other in the position shown. Each jaw has a surface 194 that faces the other jaw with those surfaces diverging away from each other towards the end of the instrument to assist in the location of a band between the jaws. Attachment of a band may be caused by urging the band towards the instrument.

Whilst the present embodiments of FIGS. 15 to 32 have been described in relation to a band being caught it will be appreciated that a suture fastened to the end of a band may also be able to be caught in the holders where the embodiment does not exclude this.

The present invention has been described in relation to a suture catcher at the end. It will be appreciated that the construction and operation of the instrument could be applied to any endoscopic instrument that requires to catch another part.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An endoscopic surgical instrument and a surgical device, in which one of the instrument or device includes an opening (76, 86, 96, 120, 140) at an end region and a channel (74,114) extending from the opening towards the end and the other of the instrument or device includes an end portion (78, 82, 90, 98, 110, 124) arranged, in use, to be at least partially located and held in the opening and a part (80,112) arranged, in use, to be at least partially located in the channel.

2. (canceled)

3. An instrument and device as claimed in claim 1 in which, in use, the end portion is arranged to be completely within the opening.

4. (canceled)

5. (canceled)

6. An instrument and device as claimed in any preceding claim 1 in which at least one of the opening and end portion or the channel and part are arranged to be a friction fit.

7. An instrument and device as claimed in claim 6 in which, in use, relative rotation of the instrument and the item is arranged to occur.

8. An instrument and device as claimed in claim 7 in which the relative rotation is arranged to be about an elongate axis of the instrument.

9. An instrument and device as claimed in claim 8 in which the end portion is part spherical.

10. (canceled)

11. An instrument and device as claimed in claim 1 in which the opening extends across the instrument or device.

12. An instrument and device as claimed in claim 1 in which the end portion is a bar.

13. (canceled)

14. An instrument and device as claimed in claim 1 in which the end portion is arranged to cooperate with the opening by relative sideways movement of the instrument and device.

15-20. (canceled)

21. A method of using an endoscopic surgical instrument and a surgical device comprising holding the instrument and device together by an end portion and a part on one of the instrument or device being at least partially located respectively in an opening at and end region and a channel extending from the opening towards the end of the other of the instrument or device.

22. A method as claimed in claim 21 comprising rotating the instrument relative to the device.

23. A method as claimed in claim 21 comprising detaching the device and instrument by pulling a projection (116) on the end portion.

24. (canceled)

25. An instrument and device as claimed in claim 1 in which there are two channels on opposed sides of the instrument or device and there are two parts on the other of the instrument or device arranged to be at least partially located in each channel.

26. An instrument and device as claimed in claim 25 in which the two parts are each connected to a single end portion.

27. An instrument and device as claimed in claim 25 in which, in use, the or each part is arranged to be completely within the channel.

28. An instrument and device as claimed in claim 25 in which the opening and the or each channel are in the instrument.

29. An instrument and device as claimed in claim 1 in which the end portion includes a projection (116) arranged, in use, to extend from the opening when the portion is located and held in the opening.
